(19) United States
(12) Patent Application Publication
(10) Pub. No.: US 2008/0159960 A1
(43) Pub. Date: Jul. 3, 2008

(54) Particles for Determining the Local Temperature

(76) Inventors: Rudiger Klingeler, Dresden (DE); Bernd Buchner, Dresden (DE); Jurgen Haase, Goppeln (DE); Albrecht Leonhardt, Dresden (DE); Axel Meye, Dresden (DE); Gerd Hammermann, Dresden (DE)

Correspondence Address: COLLARD & ROE, P.C., 1077 NORTHERN BOULEVARD, ROSLYN, NY, 11576, US

(21) Appl. No.: 11/883,593

(22) PCT Filed: Feb. 2, 2006
(86) PCT No.: PCT/EP2006/050633

§ 371 Date: Sep. 26, 2007

(30) Foreign Application Priority Data
Feb. 3, 2005 (DE) 10 2005 005 704.7

(51) Int. Cl. A61K 49/18; A61K 49/06; A61K 49/14

(52) U.S. Cl. 424/9.32; 424/9.3; 424/9.34

(57) ABSTRACT

The invention relates to particles for determining the local temperature in organic and non-organic bodies. The temperature is determined using nuclear magnetic resonance spectroscopy (NMR) when the particles are utilized. The aim of the invention is to provide temperature sensors which allow the temperature to be measured in vivo in a contactless manner on a nanometer scale while making it possible to use NMR-active substances which are foreign to the body or are contained therein in small quantities only and can be toxic, the temperature sensors allowing the temperature to be measured in vivo also on materials that are foreign to the body, e.g. nanoparticles that are heated in the body in a certain way. Another aim of the invention is to produce the temperature sensors with defined geometrical dimensions in the nanometer range. The aims are achieved by means of particles which contain a filling of one or several temperature-sensitive substances in a shell, the temperature of the substances being measurable in vivo using NMR. The shell is composed of one or several carbon nanotubes that are inserted into one another or a fullerene. Elementary metals, paramagnetic substances, materials having a magnetic order, substances having grid effects, molecular systems, and/or heterogeneous systems are selected as temperature-sensitive substances provided that the same have specific physical-chemical properties which are influenced by changes in temperature and the change in the properties of the same can be measured using NMR.

### TECHNICAL FIELD

The invention relates to particles for determining the local temperature in organic and non-organic bodies. When using these particles, the temperature is determined using the method of nuclear resonance spectroscopy (NMR).

### STATE OF THE ART

The NMR method is used in a plurality of methods for in vivo determination of the temperature. The advantage of these methods consists in the fact that the temperature can be measured in non-invasive manner and without using ionizing radiation (see, for example, U.S. Pat. No. 5,753,207, U.S. Pat. No. 5,397,562, U.S. Pat. No. 4,558,279, DE 1963 1916A1, DE 69121063T2, U.S. Pat. No. 5,711,300). No harmful side effects of the NMR method on living organisms are known.

In the case of these methods, substances are used for which the NMR signal, i.e. the spin lattice (T₁) or spin-spin (T₂) relaxation time, the chemical shift, dipolar or scalar couplings, the molecular diffusion coefficient or the equilibrium polarization in the relevant temperature range (approximately 300-370 Kelvin) changes so greatly that a determination of the local temperature is possible with sufficient accuracy (<0.1 Kelvin). An overview in this regard is given by A. G. Webb, Ann. Rep. on NMR Spectr. 45, 1 (2002).

Measurement of the NMR signal of the nuclei, in each instance, then allows the determination of the temperature in the surroundings of the corresponding substances. Medical applications, however, are essentially limited to spectroscopy of hydrogen nuclei, which frequently occur in the human body. In this connection, contrast agents are frequently used to improve the signal (DE 198 16 917 A1).

The proposal has also already been made to use carbon nanotubes filled with liquid Ga columns for determining the temperature (US 2003/0227958 A1). In this connection, a conclusion concerning the ambient temperature is to be drawn from the length change of the Ga column. However, in this connection, the question as to how these length changes are to be detected in vivo has obviously not been clarified.

In general, the NMR technique is limited to those atomic nuclei that possess a magnetic moment. Furthermore, the corresponding atomic nuclei must be present at the examination site in a correspondingly high concentration, in order to be able to generate a sufficiently strong NMR signal. Therefore, NMR spectroscopy is usually carried out on hydrogen nuclei in the magnetic resonance imaging methods (MRI) used for medical purposes, or (magnetic) contrast agents are used to reinforce the signal of protons or other NMR-active atomic nuclei inherent in the body. Specifically when using proton NMR, however, there is the difficulty of delimiting the region to be examined, since hydrogen nuclei are present everywhere in the body tissue.

Furthermore, the use of substances inherent in the body, i.e. of their atomic nuclei possesses the disadvantage of a reduced sensitivity or precision in determining the temperature, since in this connection, it is not the sensor materials having a particularly great temperature dependence, which are particularly suitable for determining the temperature by means of NMR spectroscopy, that are used.

A significant problem in the use of substances having a strongly temperature-dependent NMR signal consists in the fact that such substances have a toxic effect even in small amounts. This prohibits their use in vivo.

The use of substances inherent in the body, i.e. of their atomic nuclei, which is possible on the other hand, has the disadvantage of a reduced sensitivity or accuracy in determining the temperature, since in this connection, customized sensor materials having a particularly great temperature dependence are not used. Furthermore, there are some factors that detrimentally affect the possibilities of NMR examinations in vivo.

Furthermore, applications are possible in which heat is added to a system locally, for example by means of heating nanoparticles. With the assumption of such an introduction of energy, there is the question to what extent the power radiated in leads to heating of the nanoparticle itself, of its direct surroundings, for example of an individual cell, and of the surrounding tissue. Therefore a precise knowledge of the local temperature is necessary.

A method that uses nanoparticles for local heating in vivo, within the scope of tumor therapy, is “hyperthermia with iron oxide particles.” In this connection, temperature measurements are carried out merely on a large length scale, for example by means of the use of fiber-optic thermometers, which are applied in the tumor region.

### DISCLOSURE OF THE INVENTION

The invention is based on the task of making available temperature sensors that can be used to measure the temperature in vivo, in contact-free manner, on a nanometer scale. In this connection, the possibility of using NMR-active substances that are foreign to the body or present in the body only in small amounts, which might be toxic, is supposed to be made possible. The in vivo temperature measurement is also supposed to be possible, using the temperature sensors, on materials foreign to the body, for example on nanoparticles, which are heated in the body in a certain way. It is furthermore a task to implement these temperature sensors in the nanometer range, with defined geometrical dimensions.

This task is accomplished with the particles according to the invention, for determining the local temperature in organic and non-organic bodies, presented in claim 1. The dependent claims contain advantageous and practical forms of the invention.

The particles according to the invention are characterized in that a filling of one or more temperature-sensitive substances is contained in a shell, the temperature of which can be measured in vivo by means of NMR, wherein the shell consists of one or more carbon nanotubes (referred to hereinafter as CNTs—Nano Carbon Tubes) that are inserted into one another or of a fullerene.

By means of being embedded into the carbon shells, the temperature sensors can be applied locally and are therefore available in high concentration at the desired location. In connection with the great temperature dependence of the NMR signal of the temperature-sensitive substances, the temperature can therefore easily be determined by means of NMR.

Because of the chemically resistant shell and the mechanical stability of the CNTS, it is possible to also use temperature-sensitive substances that would have a toxic effect in the body without a protective shell. In this connection, the number of protective carbon shells can be changed by means of suitable production parameters, and adapted to the demands with regard to the required chemical and/or mechanical stability.

Elemental metals, paramagnetic substances, materials having a magnetic order, substances having lattice effects, molecular systems and/or heterogeneous systems are selected as temperature-sensitive substances, to the extent that they possess specific physical-chemical properties that are influenced by temperature changes and their property changes can be measured by means of NMR.

In this connection, it is advantageous to use copper, aluminum, tin, and rubidium as temperature-sensitive elemental metals.

The use of the elemental metals copper, aluminum, tin, and rubidium leads to an NMR signal in which the relaxation rate is greatly dependent on the temperature. When using these materials, a particularly simple selection and a simple determination of the corresponding nuclear signals is possible because of the great frequency shift.

For example, lead nitrate can be used as a temperature-sensitive paramagnetic substance.

The use of lead nitrate has the advantage of a particularly strongly temperature-dependent resonance signal, so that even very slight temperature changes can be detected rapidly and simply.

According to the invention, gadolinium, manganese arsenate, SbBr₃, CsBr₃, KBrO₃ and/or cobalt can be used as temperature-sensitive materials having a magnetic order.

These temperature-sensitive materials having a magnetic order are advantageous because in these cases, the internal magnetic fields are temperature-dependent. Therefore no additional external magnetic field has to be used to determine the nuclear resonance frequencies. Furthermore, the sensitivity of cobalt is particularly high, because of hyper-fine effects.

In particular, copper (1) oxide can be used as a temperature-sensitive substance having lattice effects.

Copper (I) oxide possesses the advantage of greatly temperature-dependent nuclear resonance frequencies, which can be determined with and without an external magnetic field. The atomic nuclei have an electrical quadrupole moment and are therefore particularly temperature-sensitive in the vicinity of the structural phase transition, i.e. in the relevant temperature range.

It is practical to use methane, propane, water and/or organic molecules as temperature-sensitive molecular systems.

According to the invention, metals and hydrogen, zeolite and water, and/or zeolite and metal can be used as temperature-sensitive heterogeneous systems.

The stated temperature-sensitive molecular systems methane, propane, water and/or organic molecules are suitable as sensors because of the temperature dependence of the movement of the molecules. It is an advantage of these compounds that the desired parameters can be adjusted in very flexible manner, since they can be easily influenced by means of appropriate mixtures.

The stated temperature-sensitive heterogeneous systems of metal and hydrogen, zeolite and water, and/or zeolite and metal offer similar flexibility.

According to a practical embodiment of the invention, one or more fullerenes having metal ions can be contained in the particles, whereby the fullerenes form the temperature-sensitive substance.

The temperature-sensitive substances that are present in the case of the fullerenes used according to the invention can be one or more of the metal ions of the metals copper and scandium and the rare earth metals, such as samarium and gadolinium.

According to an advantageous embodiment of the invention, one or more additional substances are contained in the shell of the particles, in addition to the temperature-sensitive substance.

Thus, compounds having a therapeutic and/or diagnostic effect can be contained in the shell as additional substances. According to the invention, biomolecules, elements having order numbers above 50, chromophores or fluorophores are provided as compounds having a therapeutic and/or diagnostic effect. The therapeutically effective compounds can be active substances produced in chemical or genetic manner.

Also, one or more substances suitable for hyperthermia can be contained in the shell as additional substances, or the temperature-sensitive substance can simultaneously be a substance suitable for hyperthermia. Preferably, the substances suitable for hyperthermia are ferromagnetics.

In the case of the simultaneous presence of substances having a therapeutic and/or diagnostic effect and/or of substances suitable for hyperthermia, the shells can be closed off, according to the invention, with a material that is biocompatible and degradable in the body, the dissolution of which can be monitored by the temperature sensors, under the conditions of hyperthermia, or that simultaneously allows the hyperthermia and makes it possible to monitor it.

According to a practical embodiment of the invention, biologically active carrier compounds and/or target-finding molecules can be applied to the outer surface of the particles.

The deposition of the nanoparticles according to the invention is made possible or improved at a specific location, using the target-finding molecules. The target-finding molecules can also transport the nanoparticles to a desired location. The target-finding molecules can furthermore also serve for recognizing and binding to a target molecule.

In this connection, target-finding molecules can be, among others, antibodies, antigens, special peptides, or lipids, which are attached directly or indirectly to the outer CNT shell of the nanoparticles. In this manner, the particles according to the invention represent temperature sensors that deposit locally onto certain body cells, for example, or which accumulate in a desired body region.

According to the invention, a magnetic substance can also be contained in the shell, as an additional substance. When using such particles, it is possible to control the particles in terms of their position, by means of external magnetic fields.

Also, a tracer material can be contained in the shell as an additional substance. This results in the possibility of more precise detection of the particles by means of NMR detection.

The particles according to the invention demonstrate a number of significant advantages as compared with the state of the art. In particular, they can be produced in defined manner, with regard to their geometrical dimensions. They can either be filled separately with a temperature-sensitive substance, or with additional substances and materials. The particles available for the in vivo temperature determination also demonstrate a very broad field of application, due to the use of several carbon shells, as well, and can also be bio-functionalized in targeted manner.

Fundamentally, known production and filling methods can be used for the production of the particles according to the invention, for the CNTs and the fullerenes. Thus, filling can already take place during the synthesis process, for example, specifically by means of the deposition of the particles according to the invention from the gas phase. Such a possibility has been described, for example, by A. Leonhardt et al. in “Diamond and Related Materials 3-7:790-793 (2002).”

For another thing—and this can be necessary specifically for multi-functionalized CNTs—subsequent opening and filling is also possible. In this connection, it can be desirable to dispose the CNTs to be opened on a substrate, in a well-defined orientation. The implementation of such structures is also described in detail in the literature, for example in J. Fujiwara, Journ. of Appl. Phys., 95 (2004) No. 11, p. 7118 ff. According to this reference, opening of the CNTs can take place by means of thermal treatment of the nanotubes in a defined oxygen/argon atmosphere. Another possibility for opening the nanotubes in targeted manner consists of plasma chemistry treatment in a DC-PACDV system (Direct Current—Plasma Assisted CVD). For example, it is known for H₂O plasmas that CNTs can be opened with them (L. Dai, A. Patil. Molecular Nanostructures: XVII^{th} International Winterschool/Euroconference on Electronic Properties of Novel Materials, H. Kuzmany, M. Mehring, S. Roth (eds.), AIP Conference Proceedings (2003) 621). A possible variant for opening consists in the use of an ultra-microtome.

Subsequent to being opened, the CNTs can be filled, whereby if applicable, fillings already present must be partially removed in the case of multi-functionalization. Here, a chlorine plasma can partially remove an existing Fe filling, for example. This creates the room for filling the shortened nanotubes with an additional temperature-sensitive or NMR-active agent. Afterwards, the temperature-sensitive materials can be deposited onto the substrate and thereby partially into the opened nanotubes, by means of vapor deposition in a vacuum, among other things. Afterwards, the opened CNTs are closed again. This can be done in simple manner, by means of heating. As variants, a polymer or a metal can also be applied to the open ends of the CNTs, by means of suitable deposition methods. The modified CNTs are released from the substrate by means of known chemical etching methods.

The bio-functionalization of the particles according to the invention at their outer surface is carried out using known methods. These are described in detail in EP 0625055, for example.

The corresponding functional groups is attached to the carbon shell of the CNTs, as described in V. Georgakilas, K. Kordatos, M. Prato, D. M. Guldi, M. Holzinger, A. Hirsch, J. Am. Chem. Soc. 124, 760 (2002), for example. If one opens the CNTs at one end, for example, there is a very good binding possibility for a carboxyl group, for example. The actual functionalization takes place in the next step, in which bio-functional groups bind to these carboxyl groups. CNTs functionalized with various amines have already been prepared and characterized in this manner (S. S. Wong, E. Joselevich, A. T. Wooley, C. L. Cheung, C. M. Lieber, Nature 394, 52 (1998)).

Functionalization can also be based on the method of internalization that has already been developed, by means of mediation of cationic lipids (I. Mönch, A. Meye, A. Leonhardt, K. Krämer, R. Kozhuharova, T. Gemming, M. P. Wirth, B. Büchner, Ferromagnetic filled carbon nanotubes and nanoparticles: Synthesis and lipid-mediated delivery into human tumor cells. J. Magn. Magn. Mat. (submitted)). Here, it was possible for CNTs/CNPs to penetrate into a tumor cell with the aid of the cationic lipid lipofectin, and for them to be detected cytoplasmatically. A goal is bio-functionalization of the CNTs with specific antibodies, which couple to specific surfaces of tumor cells. Furthermore, potentiation of a desired anti-proliferative effect by way of a temperature-sensitive CNT container is possible.

The temperature sensor is either supposed to be directly connected with these nanoparticles, or the body is not supposed to be able to differentiate the sensor from these nanoparticles, so that a mixture of temperature sensors and other nanoparticles can perform their function in the body, in direct proximity with one another.

### EMBODIMENTS OF THE INVENTION

In the following, the invention will be described in greater detail using exemplary embodiments.

### Example 1

This example relates to particles for determining the local temperature in the body of living beings and in non-organic materials. The particles can also be used for hyperthermia in the body of living beings. The particles consist of carbon nanotubes that are filled with cobalt as the temperature-sensitive substance and iron as the substance suitable for a hyperthermia application.

The production of these particles takes place by means of the growth of carbon nanotubes on a substrate. An Si wafer having an SiOₓ layer with a thickness of <1 μm is used as the substrate. A cobalt layer having a thickness of 2 to 5 nm is applied on top of this, by means of a physical coating method, preferably by means of vapor deposition in a vacuum. The substrate pre-coated in this manner is introduced into a CVD reactor and subjected to thermal pre-treatment at 800° C. in an argon or argon/hydrogen stream. Alternatively, thermal/plasma chemistry pre-treatment can also be carried out in a CD plasma at 600° C.

The substrate pre-treated in this manner is then heated to 900° C. and exposed to a gaseous hydrocarbon. Here, benzene in an argon/hydrogen mixture is used as the gaseous hydrocarbon. After approximately 30 sec, the hydrocarbon feed is stopped and an aerosol that is produced by means of ultrasound treatment of a 10 wt.-% ferrocene/benzene solution is introduced into the CVD reactor with the aid of an argon/hydrogen stream.

By means of the pre-treatment of the Co-coated substrate, individual Co islands having an average size of 50-100 nm have been formed by means of coalescence; these represent the catalysts for the beginning of the carbon nanotube growth.

With the introduction of the gaseous hydrocarbon benzene, carbon nanotubes filled with cobalt begin to grow on the substrate, predominantly perpendicular to it. After this benzene/Ar/H₂ mixture is taken back, and the aerosol is introduced, the nanotubes continue to grow with an iron filling as a result of the decomposition of the ferrocene. The deposition process is terminated after 3 min, in that the aerosol is taken away and only an Ar/H₂ mixture is introduced into the reactor, and subsequently, the reactor is cooled down to room temperature. After these 3 minutes, a benzene/Ar/H₂ mixture can be introduced even after the aerosol is taken back. As a result, the nanotubes continue to grow, unfilled.

After termination of the CVD process, partially or completely filled multi-wall carbon nanotubes having a diameter of 20-60 nm and a vertical orientation relative to the substrate plane exist on the Si/SiOₓ substrate. At their end close to the substrate, they possess a cobalt filling having a length of 20-100 nm, which is followed by a ferromagnetic iron filling having a length of 200-250 nm. In the case of benzene/Ar/H₂ post-treatment, a non-filled nanotube region that is dependent on the treatment time follows, which makes available the reserve space for possible other fillings, for example with therapeutics.

Magnetic and X-ray examinations of the particles produced in this manner show that both the cobalt and the iron are present in the ferromagnetic α modification and that no alloy formation between cobalt and iron occurs as a result of the comparatively short production process. Therefore it is possible to draw direct conclusions concerning the temperature in the carbon nanotubes, in the cobalt, by means of measuring the nuclear resonance frequency in the internal magnetic field, which frequency is very sensitively dependent on the temperature.

### Example 2

This example relates to particles for determining the local temperature in the body of living beings and in non-organic materials. The particles consist of carbon nanotubes that are filled with copper as the temperature-sensitive substance and iron, which is suitable for a hyperthermia application.

For the production of these particles, an Si/SiOₓ substrate coated with iron is positioned in the reaction zone of a CVD reactor such as that also used in Example 1 and pre-treated in a 50:50 Ar/H₂ stream, thermally at 800° C., or alternatively, thermally/by means of plasma chemistry at 600° C. Subsequently, the substrate treated in this manner is heated to a deposition temperature of 900-1100° C., preferably to 900° C. When the desired temperature is reached, a 10 wt.-% ferrocene/benzene solution is injected into the reactor by way of an aerosol evaporator, and there it is transported into the reaction chamber with an Ar or Ar/H₂ stream.

Carbon nanotubes having a diameter of about 20-60 nm, which are filled with α iron, grow on the substrate pre-treated in this manner, on which iron islands having a size of 50-100 nm have formed. The carbon nanotubes are partly filled partially and partly filled completely. After a coating time of 5 min, nanotubes having a length of approximately 500-700 nm have grown, with a preferred orientation perpendicular to the substrate surface.

After 5 min, the deposition process is terminated, the feed of the ferrocene/benzene-Ar/H₂ gas mixture is terminated, and the substrate on which growth has occurred is exposed to an Ar/H₂ stream. After this 5-minute “rinsing” and a temperature reduction to 700° C., the hydrogen feed is terminated and 1 vol.-% oxygen is added to the Ar gas stream, and a DC plasma is ignited above the substrate. After approximately 5 minutes, the process is terminated by shutting off the plasma and closing the oxygen valve.

An analysis of the nanostructures under a raster electron microscope shows that as a result of the plasma treatment, the nanotubes were opened by means of removing the caps. Since at least 50% of the nanotubes that were formed are only partially filled with a iron, the cavities now present can be filled by way of a coating process. This is done in the following way: The temperature of the reactor is lowered to 150° C. After this temperature is reached, an evaporator that is thermostat-controlled to 35° C. and is filled with a metal-organic compound, specifically preferably with trimethyl venyl silyl hexafluoroacetyl acetonate Cu I, is opened, and the evaporating liquid is introduced into the reactor with Ar as a transport gas. At the temperature of 150° C., the metal-organic compound is completely decomposed, and copper deposits onto the nanotubes.

Afterwards, the coating process is terminated, the reactor is cooled under argon, and the Si/SiOₓ substrate coated with nanotubes and copper in this manner is removed from the reactor.

Subsequently, the nanotubes are dissolved from the substrate in an ultrasound bath, in alcohol solution, and dispersed in the solution. Using a magnet, the filled nanotubes can be separated from the unfilled ones, but also from the copper components that have not diffused into the nanotubes and are situated in the solution as free copper particles.

The nanotubes filled with iron and copper can subsequently be closed in aqueous solution according to known methods, by means of thermal decomposition of a polymer, for example polyethylene glycol, whereby a thin graphite film extends around the opening of the nanotubes.

1. Particles for determining the local temperature in organic and non-organic bodies, consisting of a shell and a filling contained therein, wherein the shell consists of one or more carbon nanotubes that are inserted into one another, and as a filling, one or more temperature-sensitive substances are contained, the temperature of which can be measured in vivo by means of NMR (Nuclear Magnetic Resonance).

2. Particles according to claim 1, wherein elemental metals, paramagnetic substances, materials having a magnetic order, substances having lattice effects, molecular systems and/or heterogeneous systems are selected as temperature-sensitive substances, to the extent that they possess specific physical-chemical properties that are influenced by temperature changes and their property changes can be measured by means of NMR.

3. Particles according to claim 1, wherein copper, aluminum, tin, and rubidium are used as temperature-sensitive elemental metals.

4. Particles according to claim 1, wherein lead nitrate is used as a temperature-sensitive paramagnetic substance.

5. Particles according to claim 1, wherein gadolinium, manganese arsenate, SbBr₃, CsBr₃, KBrO₃ and/or cobalt are used as temperature-sensitive materials having a magnetic order.

6. Particles according to claim 1, wherein copper (1) oxide is used as a temperature-sensitive substance having lattice effects.

7. Particles according to claim 1, wherein methane, propane, water and/or organic molecules are used as temperature-sensitive molecular systems.

8. Particles according to claim 1, wherein metals and hydrogen, zeolite and water, and/or zeolite and metal are used as temperature-sensitive heterogeneous systems.

9. Particles according to claim 1, wherein the shell of the particle contains one or more fullerenes having metal ions, whereby the fullerenes form the temperature-sensitive substance.

10. Particles according to claim 1, wherein the fullerenes contain one or more of the metal ions of the metals copper and scandium and the rare earth metals, such as samarium and gadolinium.

11. Particles according to claim 1, wherein not only the temperature-sensitive substance but also one or more additional substances are contained in their shell.

12. Particles according to claim 11, wherein compounds having a therapeutic and/or diagnostic effect are contained in the shell as additional substances.

13. Particles according to claim 12, wherein biomolecules, elements having order numbers above 50, chromophores or fluorophores are contained as compounds having a therapeutic and/or diagnostic effect.

14. Particles according to claim 12, wherein the therapeutically effective compounds are active substances produced in chemical or genetic manner.

15. Particles according to claim 11, wherein one or more substances suitable for hyperthermia are contained in the shell as additional substances, or that the temperature-sensitive substance is simultaneously a substance suitable for hyperthermia.

16. Particles according to claim 15, wherein the substances suitable for hyperthermia are ferromagnetics.

17. Particles according to claim 1, wherein in the case of the simultaneous presence of substances having a therapeutic and/or diagnostic effect and/or of substances suitable for hyperthermia, the shells are closed off with a material that is biocompatible and degradable in the body, the dissolution of which can be
